(19) United States
(12) Patent Application Publication
(10) Pub. No.: US 2018/0243608 A1
(43) Pub. Date: Aug. 30, 2018

(54) SYSTEMS AND METHODS FOR BREATHING TRAINING AND METHODS TO MONITOR THEIR USE

(71) Applicant: Duke University, Durham, NC (US)

(72) Inventors: Harrison Jones, Durham, NC (US); Matt Brown, Durham, NC (US)

(21) Appl. No.: 15/900,091

(22) Filed: Feb. 20, 2018

Related U.S. Application Data
(60) Provisional application No. 62/460,951, filed on Feb. 20, 2017.

(51) Int. Cl. A63B 23/18; A61B 5/08; A61M 16/20

(52) U.S. Cl. CPC A63B 23/18; A61B 5/0816; A61B 5/486; A61M 16/208; A61B 5/0803

(57) ABSTRACT

Systems and methods for breathing training and methods to monitor their use are disclosed. According to an aspect, a breathing training monitoring system may include a gas passageway that is occluded or connected to a RMT device for receipt of input from a patient. Further, the system may include a gas pressure transducer configured to measure gas pressure within the gas passageway. The system may include a breathing activity monitor configured to determine whether a breathing activity has been accomplished based on the measure of gas pressure. The breathing activity monitor may also present an indication that the breathing activity has been accomplished in response to determining that the breathing activity has been accomplished.

### CROSS REFERENCE TO RELATED APPLICATION

This application claims priority to U.S. Provisional Patent Application No. 62/460,951, filed Feb. 20, 2017, and titled RESPIRATORY MUSCLE TRAINING MONITORING DEVICE AND METHODS OF MAKING AND USING SAME, the disclosure of which is incorporated herein by reference in its entirety.

### STATEMENT AS TO FEDERALLY SPONSORED RESEARCH

This invention was made with the support of the United States government under Federal Grant No. 1R21AR069880-01 and awarded by the National Institutes of Health (NIH). The Government has certain rights in this invention.

### TECHNICAL FIELD

The presently disclosed subject matter relates to muscle training. More particularly, the presently disclosed subject matter relates to systems and methods to provide breathing training and methods to monitor their use.

### BACKGROUND

Various devices have been developed to train the respiratory muscles for improved strength, endurance, and/or performance. Such devices are known as respiratory muscle training (RMT) devices, and they can be used to exercise the muscles of the upper airway, inspiration, and expiration by providing resistance against breathing. They can also be useful for medical diagnostics and monitoring. RMT regimens can be individualized and adjusted over time. Such devices or similarly configured devices can also be used for inspiratory muscle training (IMT) and expiratory muscle training (EMT). IMT targets the muscles of inspiration (i.e., diaphragm) via generation of negative gas pressure resistance, while EMT targets the muscles of expiration (i.e., abdominal wall) via generation of positive gas pressure resistance. RMT, IMT, and EMT may be generally referred to as breathing training.

Currently available RMT devices have substantial limitations in their ability to control important aspects of RMT dose and regimen. For example, the amount of resistance provided by flow-resistive type devices is highly dependent on flow rate, making it difficult to load the respiratory muscles systematically by providing a known exercise stimulus. Similarly, pressure-threshold devices are often not calibrated using a continuous variable or have unacceptable levels of error in terms of the actual versus intended resistance. Currently available RMT devices also provided limited feedback to the user regarding their performance with individual repetitions and training programs over time. Another limitation with current technology is the limited ability to control temporal aspects of RMT such as the duration of repetitions and the interval between them. In view of these limitations, there is a desire to provide improved RMT devices and techniques to monitor their use.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are systems and methods for breathing training and methods to monitor their use. According to an aspect, a system for breathing training may include a gas passageway for receipt of input from a patient. Further, the system may include a gas pressure transducer configured to measure gas pressure within the gas passageway. The system may include a breathing activity monitor configured to determine whether a breathing activity has been accomplished based on the measure of gas pressure. The breathing activity monitor may also present an indication that the breathing activity has been accomplished in response to determining that the breathing activity has been accomplished.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. A brief description of the drawings follows.

FIG. 1 is a block diagram of an RMT monitoring system in accordance with embodiments of the present disclosure;

FIG. 2 illustrates a flow diagram of an example method for breathing training monitoring and analysis in accordance with embodiments of the present disclosure;

FIG. 3A is a perspective view of another breathing training monitoring system in accordance with embodiments of the present disclosure;

FIG. 3B is another perspective view of another breathing training monitoring system shown in FIG. 3A;

FIG. 3C is a front view of the system shown in FIGS. 3A and 3B with the housing opened with a RMT device in place;

FIG. 3D is a front view of the system shown in FIGS. 3A and 3B with an occluded tube in accordance with embodiments of the present disclosure; and

FIG. 4A is a flow diagram of a method for RMT monitoring in accordance with embodiments of the present disclosure.

FIG. 4B is a flow diagram of a method for RMT monitoring in accordance with embodiments of the present disclosure.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

As used herein, the term “user,” “subject,” and “patient” are used interchangeably herein and refer to an individual (e.g., human) in need of, or undergoing, respiratory muscle therapy by RMT, IMT, or EMT.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the presently disclosed subject matter.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Unless otherwise defined, all technical terms used herein have the same meaning as commonly understood by one of ordinary skill in the art to which this disclosure belongs.

As referred to herein, the term “computing device” should be broadly construed. It can include any type of device including hardware, software, firmware, the like, and combinations thereof. A computing device may include one or more processors and memory or other suitable non-transitory, computer readable storage medium having computer readable program code for implementing methods in accordance with embodiments of the present disclosure. A computing device may be, for example, a server. In another example, a computing device may be a mobile computing device such as, for example, but not limited to, a smart phone, a cell phone, a pager, a personal digital assistant (PDA), a mobile computer with a smart phone client, or the like. A computing device can also include any type of conventional computer, for example, a laptop computer or a tablet computer. A typical mobile computing device is a wireless data access-enabled device (e.g., an iPHONE® smart phone, a BLACKBERRY® smart phone, a NEXUS ONE™ smart phone, an iPAD® device, or the like) that is capable of sending and receiving data in a wireless manner using protocols like the Internet Protocol, or IP, and the hypertext transfer protocol, or HTTP. This allows users to access information via wireless devices, such as smart phones, mobile phones, pagers, two-way radios, communicators, and the like. Typically, these devices use graphical displays and can access the Internet (or other communications network). In a representative embodiment, the mobile device is a cellular telephone or smart phone that operates over GPRS (General Packet Radio Services), which is a data technology for GSM networks.

The presently disclosed subject matter provides systems and method for breathing training and methods to monitor their use. The system may be used to automate collection of adherence and performance data associated with RMT. In accordance with embodiments of the present disclosure, an RMT device and/or monitoring system may include a component defining a gas passageway for receipt of input from a patient for RMT or breathing assessment. The gas passageway may be an occluded tube or a flow-resistive or pressure-threshold type RMT device. With use of an occluded tube to provide resistance against respiration, a one-way gas valve may be incorporated to allow minimal resistance against expiration during inspiratory training and minimal resistance against inspiration during expiratory training. The RMT monitoring system may also include a gas pressure transducer configured to measure positive and negative gas pressure within the gas passageway. Alternatively, for example, a flow head (pneumotachometer) and a transducer may be incorporated to measure respiratory volumes and flows. The gas pressure transducer may generate an electrical signal indicative of the gas pressure within the passageway. A breathing activity monitor may receive the electrical signal and determine whether a breathing activity has been accomplished based on the measure of gas pressure. The breathing activity monitor may also present an indication that the breathing activity has been accomplished. For example, the breathing activity monitor may include a user interface, such as a display or speaker, for presenting the indication.

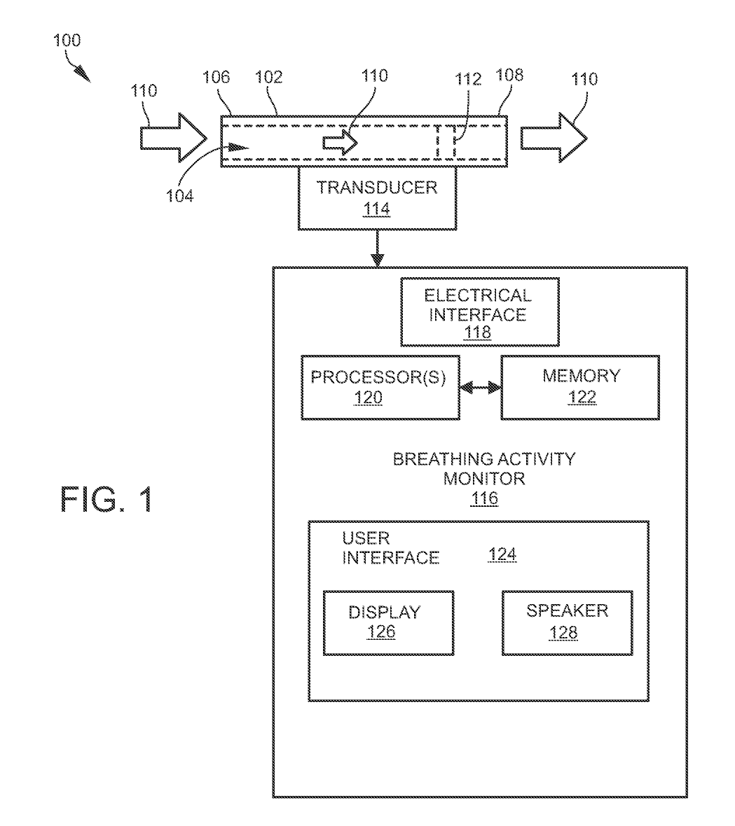

FIG. 1 illustrates a block diagram of an RMT monitoring system 100 in accordance with embodiments of the present disclosure. Referring to FIG. 1, the system 100 may include a component 102 that defines a gas passageway 104 therein for receipt of input from a patient for RMT or breathing assessment. The component 102 also includes an input end 106 and an output end 108 for passage into the component 102 and out of the component 102, respectively. The arrows 110 indicate the direction of gas flow. The input end 106 may be a mouthpiece, mask, or other suitable interface for receipt of an exhale from a patient or other user. During operation, the input end 106 may be a patient or other user may exhale into the mouthpiece such that his or her breath moves through the gas passageway 104 and exits the component 102 through the output end 108. Conversely, the patient may inhale at the input end 104 such that gas passes through the gas passageway 104 in a direction that opposes the direction of arrows 110. The patient may either inhale or exhale into the input end 106 depending on his or her training regimen.

In some embodiments, the flow of the patient's breath through the gas passageway 104 may be restricted by a feature 112 positioned within the gas passageway 104 or by a feature to occlude the gas passageway to provide resistance against inspiration or expiration. It is also noted that although the gas passageway 104 is shown as being straight in FIG. 1, the gas passageway 104 may have any suitable shape or size to achieve a desired RMT functionality.

The RMT monitoring system 100 may include a gas pressure transducer 114 configured to measure gas pressure within the gas passageway 104. Particularly, the gas pressure transducer 114 may be suitably integrated with the gas passageway 104 for measuring pressure of gas within the gas passageway 104 at one instance in time or over a period of time. As the patient exhales or inhales into the gas passageway 104 the gas pressure transducer 114 may generate an electrical signal representative of the gas pressure at any instance in time or over a period of time within the gas passageway 104. The gas pressure transducer 114 may be operably coupled with a breathing activity monitor 116 such that the electrical signal representative of the gas pressure can be communicated to the breathing activity monitor 116.

The gas pressure transducer 114 may be any transducer that is capable of converting pressure into an analog electrical signal. In some embodiments, the gas pressure transducer 114 can determine pressures between about −50 kPa to about 50 kPa. Example gas pressure transducers include the pressure sensors available from Omron Corporation (e.g., the Omron Corporation sensor having part number 2SMPP-03). Other suitable pressure sensors may also include differential and/or gage sensors.

The breathing activity monitor 116 may be any suitable computing device configured to receive an electrical input signal, to analyze the signal data, and to present analysis results. In accordance with embodiments, the breathing activity monitor 116 may include an electrical interface 118 configured to receive the electrical signal from the gas pressure transducer 114. The electrical interface 118 may be configured to condition the received electrical signal for analysis by hardware, software, firmware, or combinations thereof of the breathing activity monitor 116. For example, the breathing activity monitor 116 may include one or more processors 120 and memory 122 for processing the signal data. The breathing activity monitor 116 may also include any other suitable hardware, firmware, and/or software for implementing the functionality described herein.

The breathing activity monitor 116 may include a user interface 124 for receipt of input from a user and for presentation of information to the user. For example, the user interface 124 may include a display 126 (e.g., touchscreen display), speaker 128, and/or any other suitable components for receiving input from a user and for presentation of information to the user.

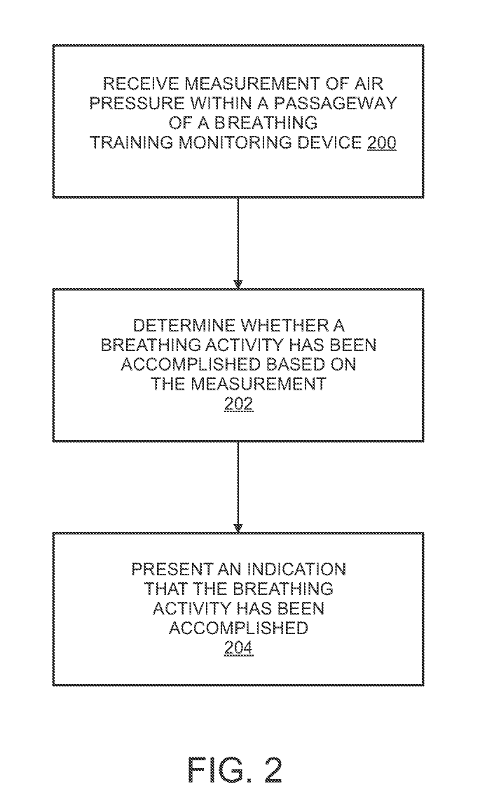

FIG. 2 illustrates a flow diagram of an example method for breathing training monitoring and analysis in accordance with embodiments of the present disclosure. It is noted that the method of FIG. 2 is described by example as being implemented by the system 100 shown in FIG. 1, although it should be understood that the method may be implemented by another suitably configured system having a transducer for measuring gas pressure within an gas passageway of RMT monitoring equipment and for presenting analysis information of the measurement to a user.

Referring to FIG. 2, the method includes receiving 200 measurement of gas pressure within a passageway of a breathing training monitoring device. For example, with reference to FIG. 1, a patient may exhale into and/or inhale from the component 102. As a result, the gas pressure within the gas passageway 104 may change. The gas pressure transducer 114 may measure the gas pressure as it changes over time, and generate an electrical signal representative of the measurement. The gas pressure transducer 114 may output the electrical signal, and the electrical interface 118 of the breathing activity monitor may receive the electrical signal. The electrical interface 118 may convert the signal to suitable form for processing by the processor(s) 120 and memory 122. In this way, the processor(s) 120 and memory 122 may receive the measure of gas pressure within the gas passageway 104.

The method of FIG. 2 includes determining 202 whether a breathing activity has been accomplished based on the measurement. Continuing the aforementioned example of FIG. 1, the processor(s) 120 and memory 122 of the breathing activity monitor 116 may analyze the data received from the electrical interface 118 for determining whether a breathing activity has been accomplished. For example, the memory 122 may store criteria for use in determining whether a breathing activity has been accomplished. The criteria may be compared to the data received from the electrical interface. Based on the comparison, the processor(s) 120 and memory 122 may determine that a particular breathing activity or set of activities has been accomplished by the patient. Example breathing activities include, but are not limited to, the patient inhaling or exhaling to a predetermined pressure level, maintaining a pressure level for a predetermined duration, performing a series of inhales and/or exhales each at a predetermined pressure level, and completion of rest periods between individual or groups of inhalations and/or exhalations. In terms of ranges, RMT can take place from about 0-300 cm H20 pressure. Regimens may vary considerably but individuals may be required to complete 5 sets of 5 expiratory repetitions per day 5 times per week. In other example, individual regimens may include 3 sets of 25 inspiratory repetitions in groups of 5 and 3 sets of 25 expiratory repetitions in groups of 5. Various short and long rest periods may be part of a regimen. A short (e.g., about 30 seconds) break required after 5 repetitions and a long break (e.g., about 10 minutes) after completing 25 before proceeding to next set of 25. The processor(s) 120 and memory 122 may, for example, determine whether the breathing activity is accomplished based on a count of respiratory muscle training repetitions and a timing of respiratory muscle training repetitions.

The method of FIG. 2 includes presenting 204 an indication that the breathing activity has been accomplished. Continuing the aforementioned example of FIG. 1, the processor(s) 120 and memory 122 of the breathing activity monitor 116 may control the user interface 124 to present an indication that the breathing activity has been accomplished. For example, the user interface 124 may provide any suitable indication that one or more breathing activities have been accomplished. More particularly, the display 126 may indicate the accomplishment by text, a graphic, or other visual signal (e.g., a red of green light). The speaker 128 may provide an audible indication of the accomplishment. The user interface 124 may also present more specific information about the breathing activity such as, but not limited to, the inhaling or exhaling pressure levels achieved by the patient and the number of repetitions of inhale or exhale. In examples, summary data of results from RMT sessions can be provided in graphic and/or tabular form.

In accordance with embodiments, the breathing activity monitor 116 may be a smartphone, tablet computer, or laptop computer, for example, configured to receive from the gas transducer 114 a signal representative of the gas pressure. For example, the signal may be received by either wired or wireless communication. An example of wireless communication includes, but is not limited to, a BLUETOOTH wireless communication technique. Further, the smartphone, tablet computer, or laptop computer may include an application residing thereon for receiving the signal data, analyzing the signal data, and presenting the analysis results to the user.

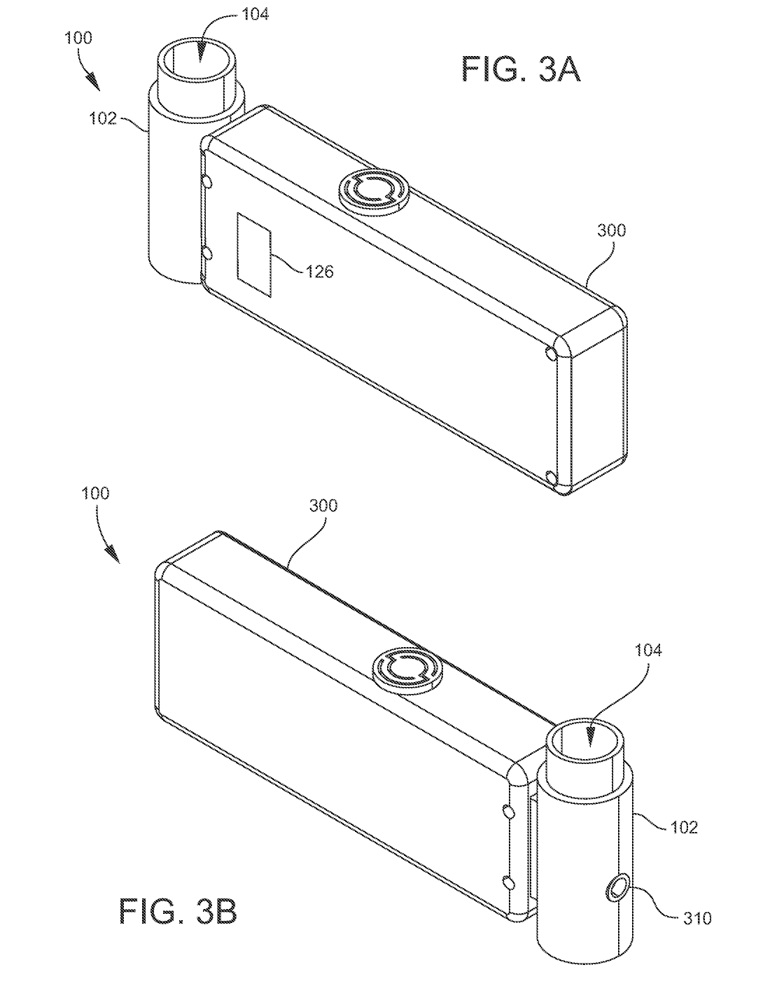

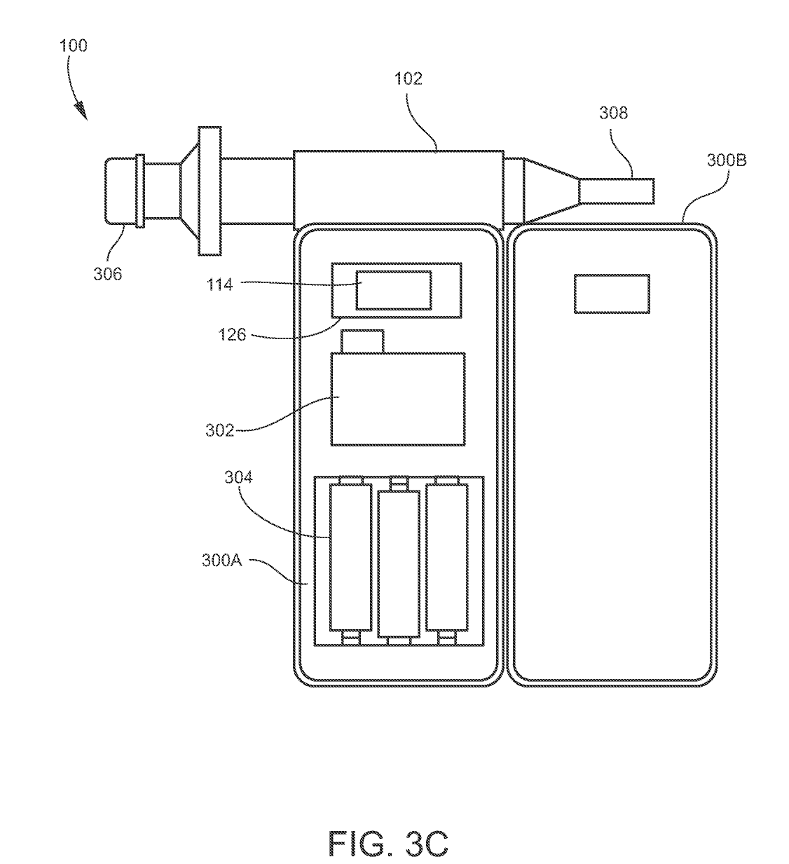

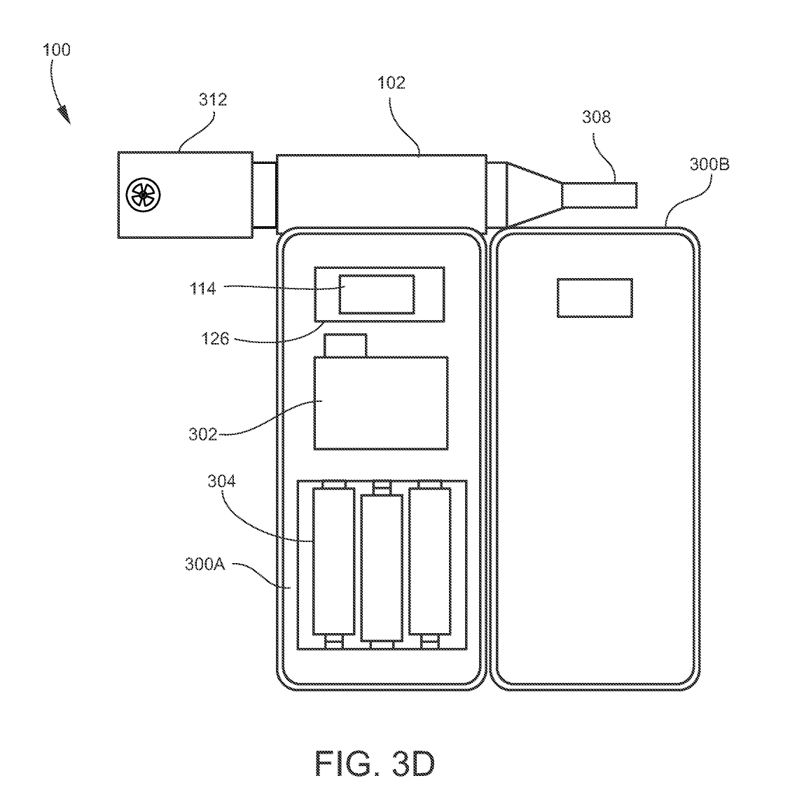

FIGS. 3A-3C illustrate different views of another breathing training monitoring system 100 in accordance with embodiments of the present disclosure. Referring to FIGS. 3A and 3B, the figures illustrate different perspective views of the system 100. The system 100 includes a housing 300. The housing 300 may be suitably sized and shaped for holding by the user. The housing 300 may define a window within which a display 126 may be fitted. The housing 300 may define an interior space and have features for containing and holding components of a breathing activity monitor as described by examples herein. The housing 300 may protect such components from damage that could result in a drop or its surrounding environment. The housing 300 may be of made of materials including, but not limited to, plastics, metals, the like, and combinations thereof.

The system 100 may also include a component 102 that defines a gas passageway 104 for receipt of inhale or exhale of a patient. One or more ends of the component 102 may be adapted to fit to other components of a breathing training monitoring equipment, such as a mouthpiece. The component 102 may also be configured to hold or otherwise interface with a gas pressure transducer (not shown in FIGS. 3A and 3B) such that the gas pressure transducer can obtain gas pressure readings from within the gas passageway 104.

FIG. 3C illustrates a front view of the system 100 with the housing 300 shown in FIGS. 3A and 3B opened to its components 300A and 300B for view and access of components inside the housing. Referring to FIG. 3C, the housing 300 contains a gas pressure transducer 114 in electric communication with a microprocessor 302 configured to determine whether a breathing activity has been accomplished based on a measurement of gas pressure within the gas passageway 104, and to control the display 126 to display an indication that the breathing activity has been accomplished in accordance with embodiments disclosed herein. The housing 300 may also contain batteries 304 or another power supply for powering the microprocessor 302, the display 126, and the transducer 114. The display 126 may be an LCD type display. In some embodiments, the system 100 may include an auditory output device, such as a speaker or piezoelectric buzzer (shown above—it is the black knob left of center and just below 2). The auditory and visual display allows for auditory and visual feedback about the patient's RMT repetitions and performance data. In some embodiments, the system 100 may include a shutoff switch for turning on or off power provided by the batteries 304 to thereby turn off or on the system.

The microprocessor 302 may be any microprocessor that can collect, analyze, and compute data receiver from a gas pressure transducer. Example microprocessors include, but are not limited to, RASPBERRY PI computing equipment, ARDUINO UNO computing equipment, BEAGLEBONE BLACK computing equipment, BANANA PI computing equipment, PANDABOARD computing equipment, LINKSPRITE PCDUINO computing equipment, INTEL GALILEO GEN 2 computing equipment, INTEL NUC series computing equipment, PARTICLE PHOTON computing equipment, and the like. In some embodiments, the user is able to program or otherwise set an intended resistance target (in cm H₂O) or other criteria via USB or other electrical communication means (e.g., wireless communication). This may be measured with the gas pressure transducer during RMT repetitions. Both flow-resistive and pressure threshold devices can be used to achieve the target load.

Alternative to the batteries 304, the microprocessor 302, transducer 114, and display 126 may be powered by any other suitable technique. In an example, the power may be provided by any suitable technique that allows for portable movement, and may such as the shown batteries, rechargeable batteries, and the like. It is within the scope of the present disclosure that the system 100 may also operate via direct electrical connection for power (e.g., plugin).

In embodiments according to the present disclosure, the system 100 may include a shutoff feature in order to provide control over other aspects of RMT regimens, such as its distribution over time. For example, this feature can be engaged to automatically turn off the system if patients or users do not wait a required interval of time between training sets, or if they have already completed their maximum RMT training dose for a period of time. For example, the patient may be asked to perform three sets of 25 RMT repetitions 5 days per week. The shutoff feature engages if the patient turns on the device too early, or if the patient has already completed his or her daily/weekly RMT dose. Such functionality can allow for more individualized RMT regimens which may influence therapeutic benefit and, more importantly, safety. For example, in some patient populations, adverse events may result from overtraining and these features can allow for control to prevent this from occurring.

In accordance with embodiments, breathing training monitoring systems disclosed herein may integrate with commercially available, commonly used RMT devices to allow enhanced functionality in example areas including, but not limited to, automated collection of adherence and performance data; enhanced control over RMT dose and regimen; and delivery of user feedback regarding performance. Such example components 306 and 308 are shown attached to component 102 in FIG. 3C. Further, the system may integrate with commercially available RMT devices by, for example, electrical connection (e.g., plug). In some embodiments, the connection comprises a standard 22 mm OD male to 22 mm ID female coupling or any other suitable manual connection.

In embodiments, the system 100 may include a light indicator 310 (shown in FIG. 3B) for signaling to a user about accomplishment of a breathing activity. For example, the light indicator 310 may be controlled by the microprocessor to indicate when the user completed a successful breathing repetition (e.g., at a desired pressure and/or desired duration). In an example, the light indicator 310 may be a light emitting diode (LED). In an example operation at the beginning of an exercise, the light indicator 310 may emit a red light. Subsequently, when a successful breathing repetition has been achieved, the light indicator 310 may emit a green light to signal to the user that a successful breathing repetition has been completed. As shown in FIG. 3B, the light indicator 310 is positioned on top of the component 102. This position may be desirable for ease of view of the user. It is also within the scope of the present disclosure that the light indicator 310 can be positioned at other locations on the component 102 or on the housing 300.

In accordance with embodiments, the housing 300 may be separated from the component 102. This can allow for it to be cleaned for preventing contamination between users.

FIG. 3D is a front view of the system shown in FIGS. 3A and 3B with an occluded tube 312 in accordance with embodiments of the present disclosure. The tube 312 may be coupled with one-way valves 314 to allow minimal resistance during inspiration for expiratory repetitions and minimal resistance during expiration for inspiratory repetitions.

In accordance with embodiment, the system disclosed herein may be used to: (i) program an intended resistance (in cm H₂O) and duration (in ms) target into the RMT monitoring system; (ii) attach the RMT monitoring system to various other RMT devices; (iii) perform the RMT exercise, following the prompts provide by the RMT monitoring system until complete; and (iv) download data recorded by the RMT monitoring system onto a computer or other network.

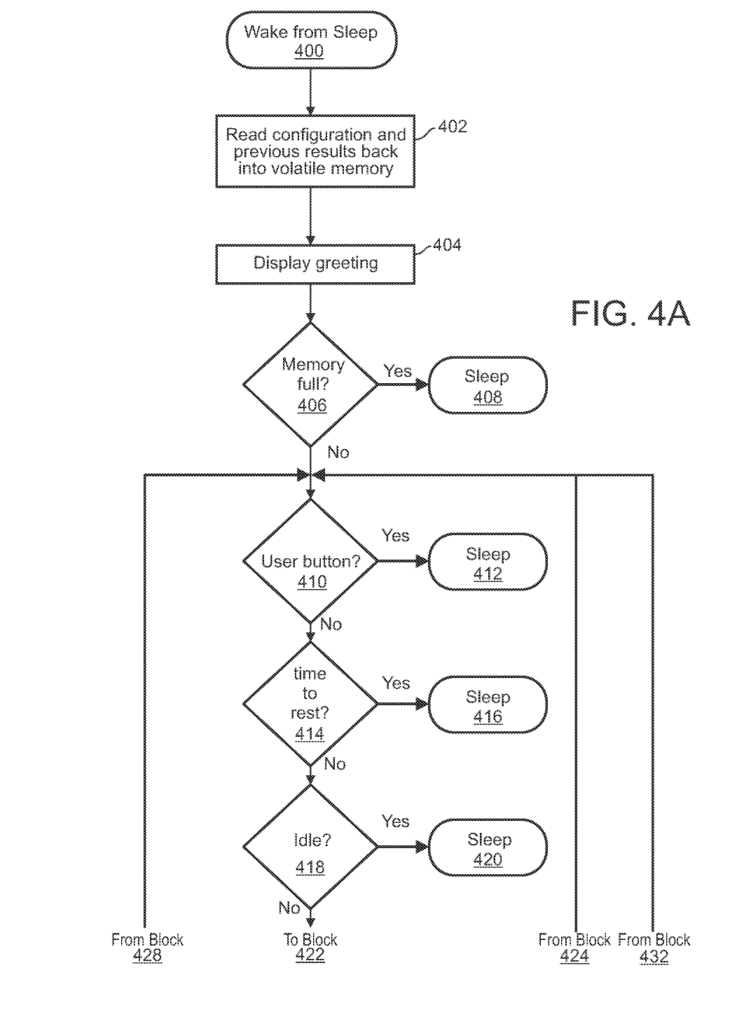

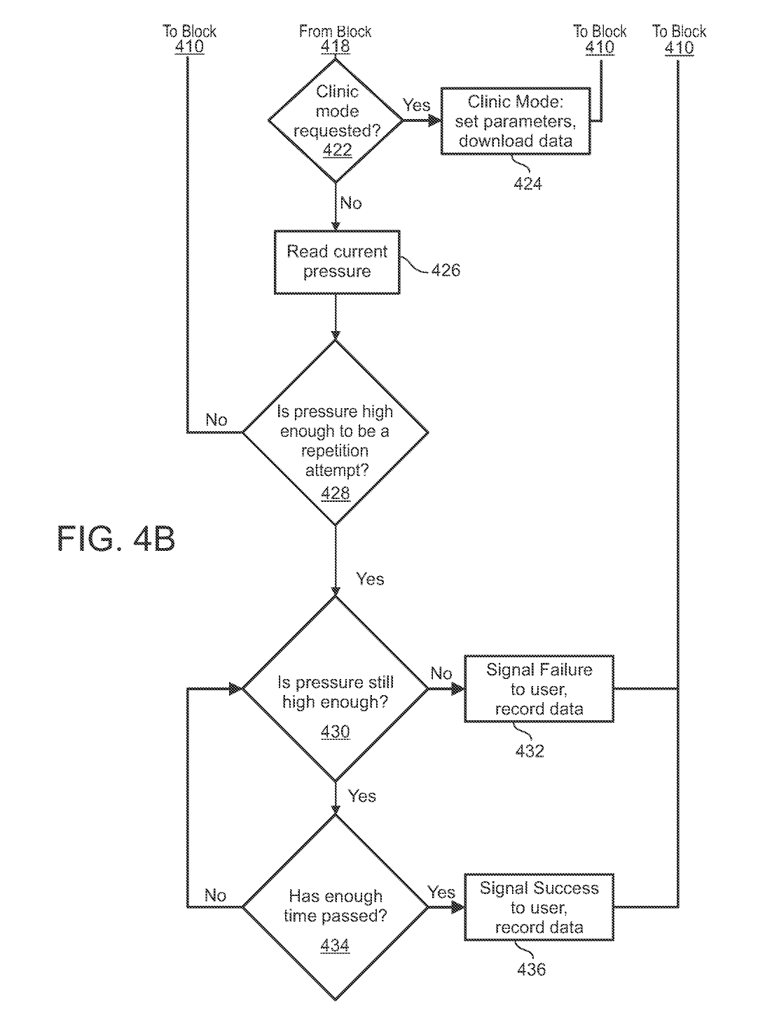

FIGS. 4A and 4B illustrate a flow diagram of a method for RMT monitoring in accordance with embodiments of the present disclosure. The method may be implement by an RMT monitoring system as disclosed herein or any other suitable monitoring system. In this example, the method is described as being implemented by the system shown in FIGS. 3A-3C. Referring to FIG. 4A, the method may start at block 400 where the system wakes from a sleep mode. Subsequently, at block 402, the microprocessor 302 may read configuration and any previous results back into volatile memory. At block 404, the microprocessor 302 may control the display 126 to display a greeting to the user. At block 406, the microprocessor 302 may determine whether memory is full. In response to determining that memory is full, the microprocessor 302 may enter a sleep mode at block 408. In response to determining that memory is not full, the microprocessor 302 may determine whether a user button is pressed or other user input is entered. If not, the microprocessor 302 may enter a sleep mode at block 412

In response to determining that the user button was pressed or other user input was entered, the microprocessor 302 can determine whether it is time to rest at block 414. In response to determining that it is time to rest, the microprocessor 302 may enter a sleep mode at block 416. In response to determining that it is not time to rest, the microprocessor 302 can determine whether to idle at block 418. In response to determining to idle, the microprocessor 302 may enter a sleep mode at block 420. As further shown in FIG. 4B, in response to determining that it is not time to idle, the microprocessor 302 can determine whether a clinic mode has been requested at block 422. In response to determining that a clinic mode has been requested, at block 424 the clinic mode may be entered where parameters are set and data downloaded.

In response to determining that the clinic mode has not been requested, the microprocessor 302 can read a current pressure from the gas pressure transducer 126. As shown in FIG. 4B at block 428, the microprocessor 302 can determine whether the read pressure is high enough to be a repetition attempt. In response to determining that the read pressure is not high enough, the process may return to block 410. Otherwise, in response to determining that the read pressure is high enough to be a repetition attempt, the microprocessor 302 may determine whether the pressure is still high enough at block 430. If it is determined the pressure is not still high enough, at block 432 a signal failure is indicated to the user by use of the display 114 and the data is recorded in memory. In response to determining that the pressure is still high enough, the method may proceed to block 434.

At block 434 of FIG. 4B, the microprocessor 302 may determine whether enough time has passed. In response to determining that enough time has not passed, the method may proceed back to block 430. In response to determining that enough time has passed, a signal success is indicated to the user by use of the display 114 and the data is recorded in memory at block 436. Subsequently, the method returns to block 410.

In example uses of a system as disclosed herein, a RMT therapy sessions may be conducted by the RMT clinician and each visit may take approximately 45 minutes. Therapy visits can include the following sequential steps:
Measurement of MIP and MEP by the RMT clinician. MIP and MEP measurement will occur at the beginning of each RMT therapy session in addition to the 4 assessments previously described. This allows the RMT clinician to provide progressive resistance for subjects in both study arms.
Download and review of RMT adherence and performance data from data collection/dose control/feedback tool via USB.
Calibration of RMT pressure-threshold device and programming of data collection/dose control/feedback tool.
Completion of 1 set of 25 repetitions for each inspiratory and expiratory RMT/sham-RMT; behavioral observations will be made by clinician of RMT/sham-RMT tolerance throughout.
Subjects may be asked to rate pain and perceived effort associated with RMT using a standard 0-10 scale, intermittently and after each RMT/sham-RMT set.
Based on results of steps 4 and 5, the RMT/sham-RMT training stimulus will be modified (details below) if pain rating is >4, perceived effort rating is >8, and/or behavioral observations suggest excessive effort. Additionally, in the control arm only, the sham-RMT training stimulus may be modified if subjects exhibit minimal effort suggestive of attempts by the subject to penetrate the blind.
Completion of alternating sets of 25 repetitions of inspiratory and expiratory RMT/sham-RMT while steps 5 & 6 are repeated until 3 well-tolerated sets of 25 successful inspiratory and expiratory RMT/sham-RMT repetitions are achieved.

It is noted that memory disclosed herein may alternatively be referred to a computer readable storage medium. A computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A method comprising:
receiving measurement of gas pressure within a passageway of a breathing training monitoring device or occluded tube;
determining whether a breathing activity has been accomplished based on the measurement; and
in response to determining that the breathing activity is met, presenting an indication that the breathing activity has been accomplished.

2. The method of claim 1, wherein receiving the measurement of gas pressure comprises receiving an electrical signal representative of the gas pressure within the gas passageway or the occluded tube.

3. The method of claim 1, further comprising:
determining, based on the measurement of gas pressure, at least one of a count of breathing training repetitions and a timing of breathing training repetitions; and
wherein determining whether the breathing activity has been accomplished comprises determining whether the breathing activity has been accomplished based on the determined at least one of the count of breathing training repetitions and a timing of breathing training repetitions.

4. The method of claim 1, wherein presenting the indication comprises using a user interface to present the indication that the breathing activity has been accomplished.

5. The method of claim 4, wherein using a user interface comprises using at least one of a display and a speaker.

6. The method of claim 1, further comprising using at least one processor and memory for determining whether a breathing activity has been accomplished.

7. The method of claim 1, further comprising using a gas pressure transducer to obtain the measurement of gas pressure.

8. The method of claim 1, further comprising wirelessly communicating the received measurement to a computing device located remote from the breathing training monitoring device, and
wherein the method comprises using at least one processor and memory of the computing device for determining whether the breathing activity has been accomplished, and for presenting the indication that the breathing activity has been accomplished.

9. A breathing training monitoring system comprising:
an occluded tube or a gas passageway for receipt of input from a patient;
a gas pressure transducer configured to measure gas pressure within the gas passageway or the occluded tube; and
a breathing activity monitor configured to:
determine whether a breathing activity has been accomplished based on the measure of gas pressure; and
present an indication that the breathing activity has been accomplished in response to determining that the breathing activity has been accomplished.

10. The system of claim 9, wherein the gas pressure transducer is configured to generate an electrical signal representative of the gas pressure within the gas passageway or the occluded tube.

11. The system of claim 9, wherein the breathing activity monitor is configured to:
determine, based on the measurement of gas pressure, at least one of a count of breathing training repetitions and a timing of breathing training repetitions; and
determine whether the breathing activity has been accomplished based on the determined at least one of the count of breathing training repetitions and a timing of breathing training repetitions.

12. The system of claim 9, further comprising a user interface configured to present the indication that the breathing activity has been accomplished.

13. The system of claim 12, wherein the user interface comprises at least one of a display and a speaker.

14. The system of claim 9, wherein the breathing activity monitor comprises at least one processor and memory configured for determining whether the breathing activity has been accomplished.

15. The system of claim 9, further comprising a communications module configured to wirelessly communicate the received measurement to a computing device, and
wherein the computing device comprises the breathing activity monitor.
